(19) United States
(12) Patent Application Publication
(10) Pub. No.: US 2017/0172784 A1
(43) Pub. Date: Jun. 22, 2017

(54) ARTIFICIAL PENIS

(71) Applicant: ANDREAS KIEFER, BOEBLINGEN (DE)

(72) Inventor: ANDREAS KIEFER, BOEBLINGEN (DE)

(21) Appl. No.: 15/380,809

(22) Filed: Dec. 15, 2016

(30) Foreign Application Priority Data
Dec. 16, 2015 (DE) 202015008591.1

(51) Int. Cl. A61F 5/41

(52) U.S. Cl. CPC A61F 5/41; A61F 2005/411

(57) ABSTRACT

Artificial penis 1 for a male person which simulates an enlargement of the smaller erect natural penis. The artificial penis (1) has an interior recess (2) to accommodate the imaginary erect natural penis via an insertion opening (3). The recess (2) comprises a glans-shaped compartment (2a) for the accommodation of the imaginary glans of the erect natural penis, and a penis-shaft-shaped compartment (2b) for the imaginary accommodation of the shaft adjoining the glans of the erect natural penis. At the transition from the compartment (2a) to compartment (2b), a retaining bulge (4) is arranged which lies against the slope of the glans of an imaginary erect natural penis inserted into the artificial penis. The artificial penis (1) has a fastening component (1K) with a distensible elastic entry opening for part of the scrotum and the testicles, by means of which the artificial penis (1) can be fastened detachably to the human body.

This patent application claims the benefit and priority of and to German patent application serial no. No. 20 2015 008 591.1, filed Dec. 16, 2015. German patent application serial no. No. 20 2015 008 591.1, filed Dec. 16, 2015 is incorporated herein by reference hereto in its entirety.

The invention refers to an artificial penis for attachment to an erect smaller natural penis of a male person.

In order to avoid misunderstandings, it should be noted that the term “artificial penis” means a false or imitation penis.

The external sexual organs of a male are the penis and the scrotum.

The foremost part of the penis is the glans which, in the non-erect state of the penis is concealed almost entirely by the foreskin. In the erect state, the foreskin slips behind the glans by itself. The part of the penis behind the glans is known as the shaft. The upper edge of the glans falls away towards the shaft. This “falling away” is referred to here as the slope of the penis.

The purpose of the invention is to provide an artificial penis for a male person which simulates the enlargement of a smaller erect natural penis.

Additional advantages, features and possible applications of the present invention may be gathered from the description which follows, in which reference is made to the embodiments illustrated in the drawings.

Throughout the description, claims and drawings, those terms and associated reference signs are used as are listed in the List of Reference Signs below. In the drawings,

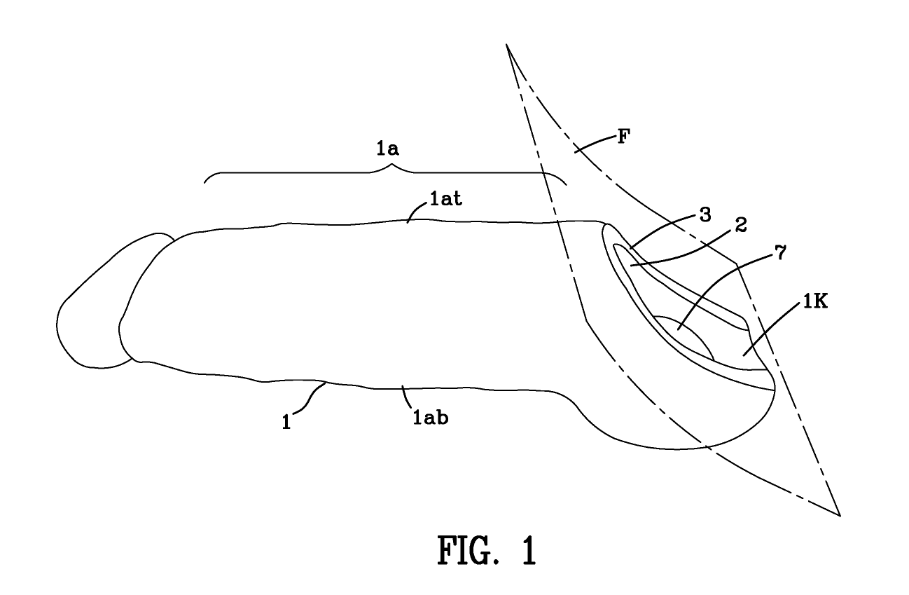

FIG. 1 a side view of the artificial penis according to the invention in isometric representation;

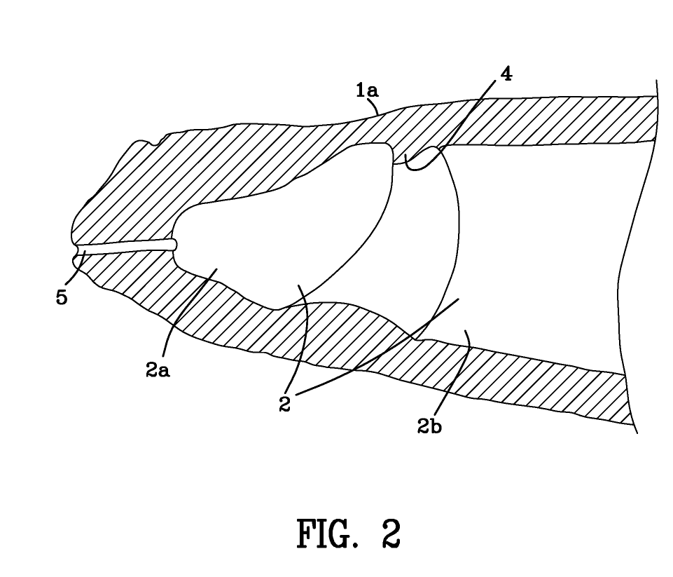

FIG. 2 a partial cross-section of the artificial penis according to the invention with sperm duct;

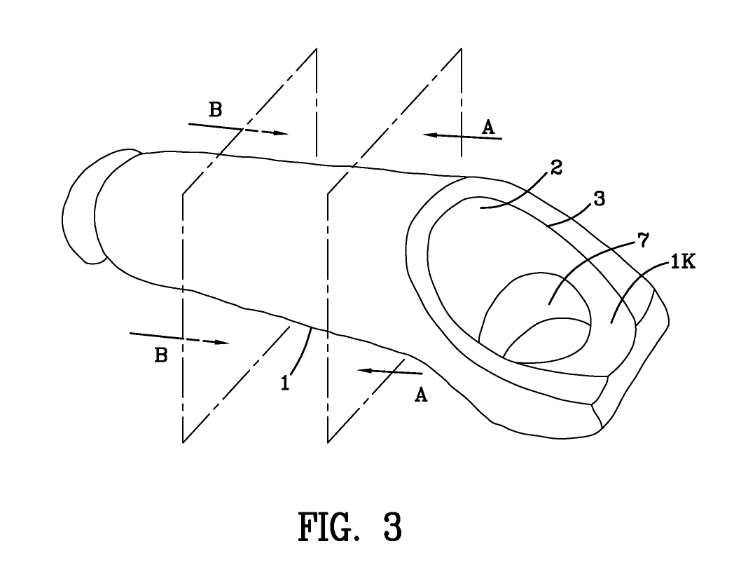

FIG. 3 another side view of the artificial penis according to the invention in isometric representation with the entire insertion opening and the fastening component viewed diagonally;

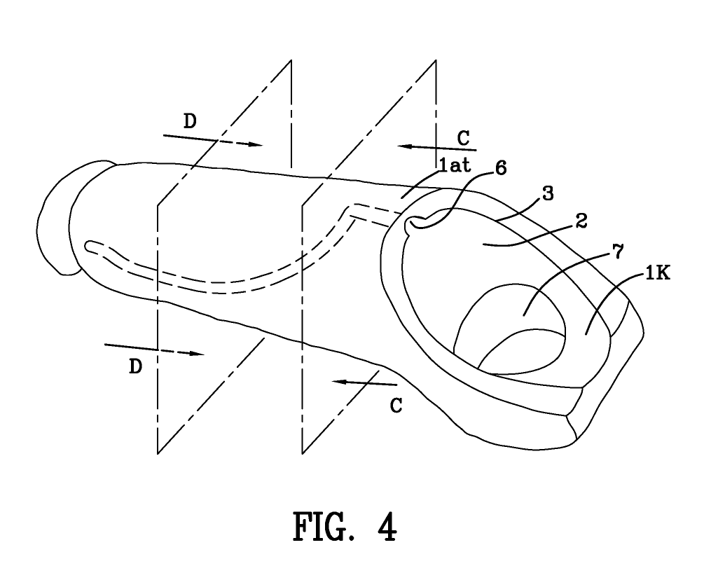

FIG. 4 the artificial penis according to the invention with a ventilation duct;

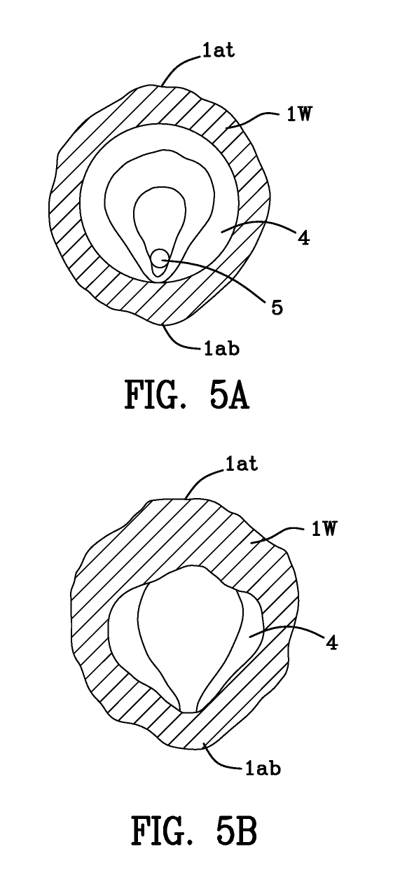

FIG. 5A a sectional view of the artificial penis according to the invention with sperm duct in accordance with the section plane A-A in FIG. 3;

FIG. 5B a sectional view of the artificial penis according to the invention with sperm duct in accordance with the section plane B-B in FIG. 3;

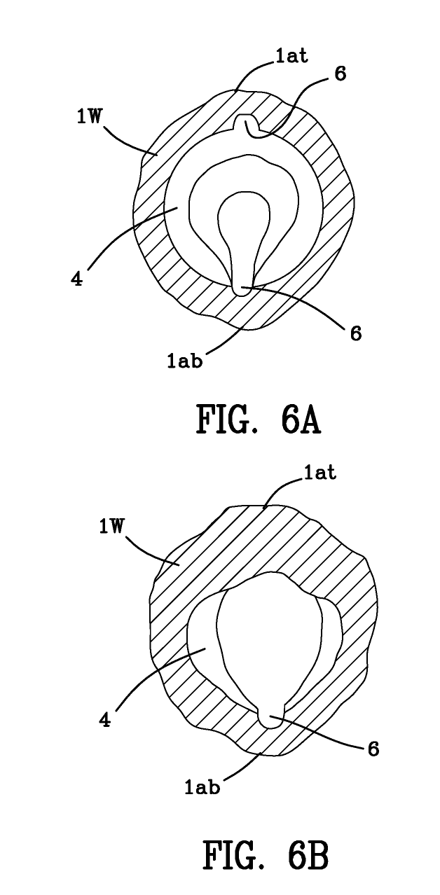

FIG. 6A a sectional view of the artificial penis according to the invention with ventilation duct in accordance with the section plane C-C in FIG. 4;

FIG. 6B a sectional view of the artificial penis according to the invention with ventilation duct in accordance with the section plane D-D in FIG. 4, and

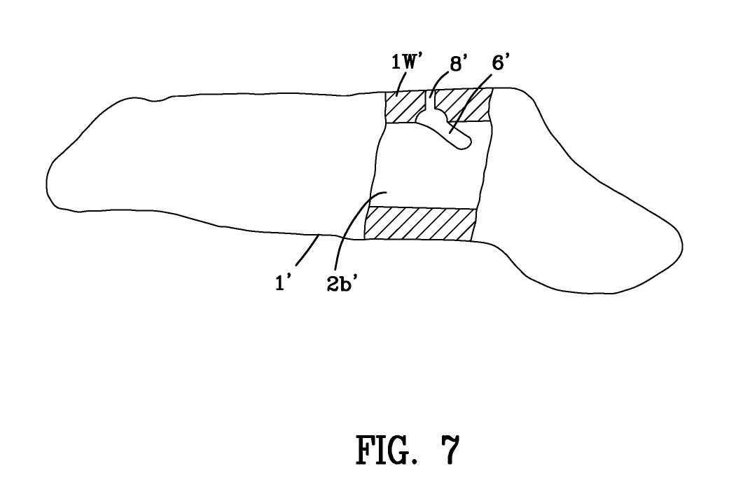

FIG. 7 a side view of the artificial penis with a partial cross-section of the artificial penis with a ventilation duct and a ventilation opening in the wall of the artificial penis.

The artificial penis is intended to simulate the increase in size of an erect natural penis during sexual intercourse. The artificial penis 1 includes a fastening component 1K by means of which it can be fastened detachably to the imaginary human body, as is described below.

The artificial penis has an interior recess 2 to accommodate the imaginary erect natural penis via an insertion opening 3.

In the foremost part of the artificial penis 1 this recess 2 comprises a glans-shaped compartment 2a for the accommodation of the imaginary glans of the erect natural penis, and, adjoining the compartment 2a in the direction of the insertion opening, a shaft-shaped compartment 2b to accommodate the shaft adjoining the glans of the erect natural penis.

In the area of the transition from the compartment 2a to compartment 2b in the interior of the recess 2, there is a bulge 4 which lies against the slope of the glans of an imaginary erect natural penis inserted into the artificial penis, thereby giving the artificial penis a degree of hold at its foremost part to prevent it from slipping off. The artificial penis 1 is made of elastic material, preferably silicone.

In the upper area 1at of the artificial penis 1 the retaining bulge 4 is higher and more narrow than in the lower area 1ab of the artificial penis 1 (see also FIGS. 5A, 5B, 6A, 6B).

The fastening component 1K is arranged near the insertion opening 3. The insertion opening (3) has a distensible elastic entry opening 7. When suitably distended, the entry opening (7) permits insertion of part of the scrotum with the testicles, thereby effecting the attachment of the artificial penis 1 to the human body.

This attachment can be released by withdrawing the inserted part of the scrotum and the testicles from the opening 7 by means of stretching the entry opening 7.

The insertion opening 3 is elliptical in shape. It is located in an imaginary arched surface F which runs diagonally and slightly arched to the hollow-cylinder-shaped shaft 1a of the artificial penis 1. The entry opening 7 is located in the wall of the lower part 1a of the shaft 1a of the artificial penis 1 onto which the insertion opening 3 can be projected.

Expressed simply, the area of the insertion opening 3 of the artificial penis is comparable to a diagonal cut of the end of a horizontal tube, where the upper edge of the opening does not cover the lower edge of the opening.

In particular, FIG. 2 shows the division of the inner recess 2 into the glans-shaped compartment 2a, the shaft-shaped compartment 2b and the arrangement of the retaining bulge 4. This construction is common to two embodiments of the invention. As shown in FIG. 2, the first embodiment also has a sperm duct 5 while the second embodiment as seen in FIG. 4 also has a ventilation duct 6.

As seen in FIG. 2, the sperm duct 5 (which permits the discharge of the ejaculate as well as ventilation) extends from the foremost part of the glans-shaped compartment 2a and issues from the foremost part of the glans of the artificial penis 1.

FIG. 4 shows an isometric representation of the embodiment of the artificial penis 1 with a ventilation duct 6 as in FIG. 3. The ventilation duct 6 runs in the wall of the compartments 2a and 2b to the insertion opening 3. It commences in the foremost part of the glans-shaped compartment 2a and extends in a spiral to the upper area 1at of the penis shaft 1a and issues at the insertion opening 3. The ventilation duct 6 is open towards the recess 2. Its profile may be of any shape.

FIG. 5A shows a sectional view of the artificial penis 1 with sperm duct 5 in accordance with the section plane A-A in FIG. 3. The sectional view is viewed in direction A. The wall of the artificial penis 1 is indicated as 1w, the retaining bulge as 4 and the sperm duct as 5.

FIG. 5B shows a sectional view of the artificial penis 1 with sperm duct in accordance with the section plane B-B in FIG. 3. The sectional view is viewed in direction B. The wall of the artificial penis 1 is indicated as 1w and the retaining bulge as 4.

FIG. 6A shows a sectional view of the artificial penis 1 with ventilation duct in accordance with the section plane C-C in FIG. 4. The sectional view is viewed in direction C. The wall of the artificial penis 1 is indicated as 1w, the retaining bulge as 4 and the ventilation duct as 6.

FIG. 6B shows a sectional view of the artificial penis 1 with ventilation duct in accordance with the section plane D-D in FIG. 4. The sectional view is viewed in direction D. The wall of the artificial penis 1 is indicated as 1w, the retaining bulge as 4 and the ventilation duct as 6.

In FIGS. 5A, 5B, 6A and 6B, the upper part of the artificial penis is indicated as 1 at and the lower part as 1ab.

FIG. 7 shows a side view of the artificial penis 1′ with a partial cross-section of the artificial penis and a ventilation duct 6′ and a ventilation opening 8′ in the wall 1w′ of the artificial penis 1′. The imaginary cutting plane in FIG. 7 divides the artificial penis 1′ lengthwise and passes through the end area of the ventilation duct 6′ and the ventilation opening 8′.

The ventilation duct 6′ runs on the inner wall of the glans-shaped compartment for the imaginary accommodation of the glans of the erect natural penis and on the inner wall 1w′ of the shaft-shaped compartment for the imaginary accommodation of the shaft of the erect natural penis adjoining the glans.

The ventilation duct begins in the front area of the compartment for the glans and runs in a preferably spiral-shaped path to the compartment 2b′. It (6) ends at the insertion opening of the artificial penis (as shown in FIG. 4) or it (6′) ends before this, as shown in FIG. 7.

The ventilation duct 6′ is connected to the area outside the artificial penis 1′ by at least one ventilation opening 8′ in the wall 1w′ of the artificial penis 1′.

### LIST OF REFERENCE SIGNS

1, 1′ Artificial penis
1a Shaft of artificial penis
1K Fastening component
1at Upper part of artificial penis
1ab Lower part of artificial penis
1w, 1w* Wall
2 Inner recess
2a Glans-shaped compartment
2b, 2b′ Shaft-shaped compartment
3 Insertion opening
4 Retaining bulge
5 Sperm duct
6 Ventilation duct
7 Entry opening
F Arched surface
8′ Ventilation opening

1. Artificial penis characterised in that the artificial penis (1) has an inner recess (2) for the imaginary accommodation of an erect natural penis via an insertion opening (3),
that the recess (2) in the foremost part has a glans-shaped compartment (2a) for the imaginary accommodation of the glans of the erect natural penis,
that, adjoining the compartment 2a in the direction of the insertion opening (3) the recess (2) has a shaft-shaped compartment (2b) for the imaginary accommodation of the shaft adjoining the glans of the erect natural penis,
that in the area of the transition from the compartment (2a) to compartment (2b) there is a bulge (4) which lies against the slope of the glans of an imaginary erect natural penis (1) inserted into the artificial penis,
that the artificial penis (1) has a fastening component (1K) by means of which it (1) can be fastened detachably to the imaginary human body, and
that the artificial penis (1) is made of elastic material.

2. Arrangement in accordance with claim 1, characterised in that the fastening component (1K) is arranged in the edge area of the opening (3) for inserting the natural penis into the artificial penis (1) and has an elastically distensible entry opening (7) and
that when the entry opening (7) is elastically stretched, the imaginary part of the scrotum with the testicles can pass through the entry opening (7).

3. Arrangement in accordance with claim 2, characterised in that the insertion opening (3) is elliptical in shape and is located in an imaginary arched surface (F) which runs diagonally to the hollow-cylinder-like penis shaft (1a) of the artificial penis (1),
and that the entry opening (7) is arranged in the wall of the lower area (1ab) of the shaft (1a) of the artificial penis (1), onto which the insertion opening (3) can be projected.

4. Arrangement in accordance with claim 1, characterised in that a sperm duct (5) is provided extending from the foremost part of the glans-shaped compartment (2a) and issuing from the foremost part of the artificial penis (1).

5. Arrangement in accordance with claim 1, characterised in that a ventilation channel (6) is provided which runs along the inner wall of the recess compartments (2a) and (2b) to the insertion opening (3).

6. Arrangement in accordance with claim 1, characterised in that the elastic material of which the artificial penis (1) is made is silicone.

7. Arrangement in accordance with claim 1, characterised in that a ventilation duct (6′) is provided which runs on the inner wall of the compartment for the glans of the erect natural penis, and on the inner wall and of the compartment (2b′) for the shaft of the erect natural penis and is connected to the area outside the artificial penis 1′ by at least one ventilation opening 8′ in the wall 1w′ of the artificial penis 1′.
